(19) United States
(12) Patent Application Publication
(10) Pub. No.: US 2009/0048585 A1
(43) Pub. Date: Feb. 19, 2009

(54) BRUSH DEVICE FOR GRASPING AND MANIPULATING TISSUE

(76) Inventors: Wayne A. Noda, Mission Viejo, CA (US); Stephen Graham Bell, Rome (IT)

Correspondence Address: ROGITZ & ASSOCIATES, 750 B STREET, SUITE 3120, SAN DIEGO, CA, 92101, US

(21) Appl. No.: 12/191,524

(22) Filed: Aug. 14, 2008

Related U.S. Application Data
(60) Provisional application No. 60/956,089, filed on Aug. 15, 2007.

(51) Int. Cl. A61B 17/94

(52) U.S. Cl. 606/1

(57) ABSTRACT

A brush-like element can be housed within a vacuum sleeve for advancement through a natural orifice to tissue, such as an appendix or gall bladder or diverticulum, to be inverted pursuant to resolution of a malady associated with the tissue. The brush is advanced out of the sleeve into the tissue and if desired rotated, and vacuum may also be drawn through the sleeve to further grip the tissue. The brush is then retracted into the sleeve to clamp or trap the tissue for inversion or other manipulation.

This application claims priority from U.S. provisional patent application 60/956,089, filed Aug. 15, 2007.

### FIELD OF THE INVENTION

The present application relates generally to brush devices for grasping and manipulating tissue particularly for natural orifice surgery.

### BACKGROUND OF THE INVENTION

The present assignee's U.S. patent publication no. 2007/0225734 and U.S. patent application Ser. No. 11/788,597, both of which are incorporated herein by reference, disclose various natural orifice surgery systems and methods for resolving maladies such as diverticulosis and appendicitis and for removing organs such as the gall bladder. The present application is directed to structures and methods for inverting tissue particularly in connection with natural orifice surgery to facilitate the resolution of the tissue.

### SUMMARY OF THE INVENTION

An apparatus includes a tissue gripping element housed within a tubular member for advancement through a natural orifice to tissue to be inverted pursuant to resolution of a malady associated with the tissue. The tissue gripping element is advanceable out of the tubular member into the tissue to grip the tissue and is retractable into the tubular member to grip the tissue for manipulation of the tissue. The tissue gripping element includes plural discrete gripping points.

In some embodiments the gripping points can be established by ends of respective bristles oriented generally radially relative to the tubular member. In other embodiments the gripping points are established by ends of respective teeth. The teeth may be arranged on a proximal-facing transverse surface, and plural tubular member teeth can be arranged on a distal-facing transverse surface of the tubular member. The tissue gripping element is distally advanceable relative to the tubular member to space the surfaces from each other and is proximally retractable relative to the tubular member to trap tissue between the surfaces.

The tubular member may establish a vacuum lumen through which a vacuum can be drawn to attract tissue toward the tubular member. Tissue can be attracted toward an open distal end of the tubular member when a vacuum is drawn in the tubular member, and in some embodiments tissue can also be attracted toward plural vacuum openings formed in the tubular member when a vacuum is drawn in the tubular member. A cover tube may be advanced over the tubular member to block at least some of the vacuum openings.

If desired, the tissue gripping element can be rotatable relative to the tubular member. Also, a smooth rounded atraumatic surface can be provided for establishing a distal end of the tissue gripping element.

In another aspect, a method includes establishing a retracted configuration of a tissue manipulation device in which a tissue gripping element is retracted entirely within a sleeve. The tissue gripping element includes plural discrete gripping points. The method also includes providing instructions to advance the tissue manipulation device through a natural body orifice to tissue to be manipulated, providing instructions to advance the tissue gripping element out of the sleeve, and providing instructions to manipulate the tissue gripping element to grip tissue. Instructions to retract the tissue gripping element toward the sleeve can also be provided.

In another aspect, a system includes a delivery tube advanceable into a natural body orifice toward tissue to be manipulated and an elongated control rod extending from a proximal end of the tube and manipulable by a person. A tissue gripping element is coupled to the control rod and is advanceable out of a distal end of the delivery tube. The gripping element includes plural individual grippers configured to engage tissue and thereby provide a means for manipulating the tissue by manipulating the control rod.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a perspective partially schematic view of a tissue inversion apparatus, with portions broken away;

FIG. 2 is a perspective view of the distal end of an alternate tissue inversion apparatus in the extended position;

FIG. 3 is a perspective view of the distal end of a tissue inversion apparatus in the housed position for advancing the brush toward tissue to be inverted;

FIG. 4 is a perspective view of the distal end of the tissue inversion apparatus of FIG. 3, with the brush in the extended position in tissue to be inverted and after a vacuum has been established;

FIG. 5 is a perspective view of the distal end of the tissue inversion apparatus of FIG. 4, with the brush retracted part way back into the sleeve to trap tissue;

FIGS. 6-8 are perspective views of the distal segments of non-limiting implementations of brush inversion apparatus;

FIGS. 9-12 are schematic diagrams of the distal portion of an alternate tissue inversion apparatus showing how tissue is clamped between opposed jagged surfaces; and

FIG. 13 is a perspective view of the distal segment of yet another non-limiting implementation of brush inversion apparatus.

### DETAILED DESCRIPTION OF THE PREFERRED EMBODIMENT

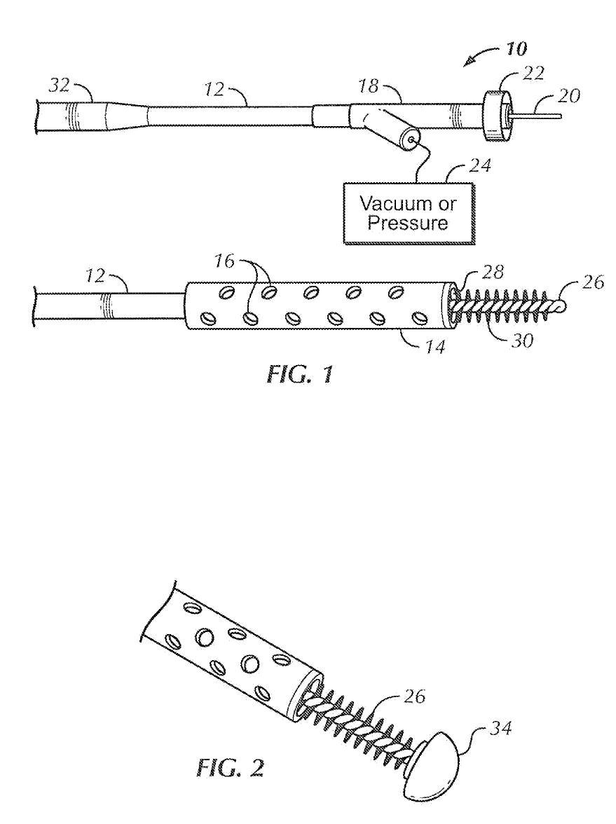

Referring initially to FIG. 1, a system is shown, generally designated 10, that includes a flexible elongated hollow sleeve-like delivery tube 12 that is advanceable into a natural body orifice such as the mouth or anus toward tissue to be manipulated. If desired, at least a distal sleeve 14 of the delivery tube 12 may be configured with plural vacuum openings 16 such as round holes, elongated slots, etc. as more fully disclosed below. The openings 16 may establish a spiral pattern as shown.

As shown in FIG. 1, the delivery tube 12 may terminate proximally at a “wye” connector 18, one branch of which supports a manipulable elongated control rod 20 around which a collet 22 may be tightened to prevent movement of the rod 20 relative to the “wye” 18. The other branch of the “wye” forms a lumen that may be connected to a pressure source 24. The pressure source 24 may be a negative pressure source (a vacuum) or a positive pressure source (such as a fluid pump or pressurized fluid source) for establishing a desired pressure within the delivery tube 12.

As also shown in FIG. 1, a tissue gripping element 26 is coupled to the control rod 20 and is advanceable out of an open distal end 28 of the delivery tube 12. In the embodiment shown in FIG. 1, the tissue gripping element 28 includes plural individual, discrete grippers 30 such as brush bristles that are configured to engage tissue and thereby provide a means for manipulating the tissue by manipulating the control rod 20. At least some of the bristles may be oriented generally radially relative to the delivery tube 12 as shown. If desired, a cover tube 32 may be advanceable over the delivery tube to block at least some of the vacuum openings 16.

FIG. 2 shows that in some implementations, a smooth rounded atraumatic surface 34 may be provided on the distal end of the tissue gripping element 26 to facilitate advancing the gripping element 26 into tissue atraumatically to the tissue.

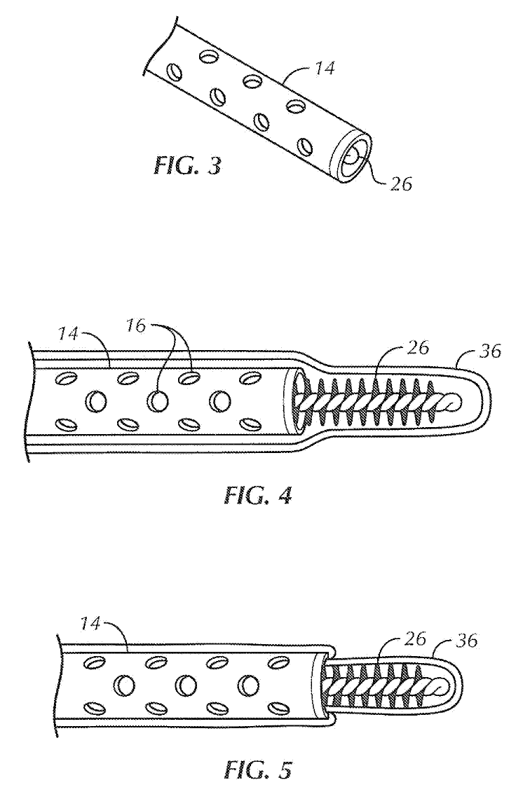

FIGS. 3-5 illustrate various operational configurations of the system 10. In FIG. 3, the gripping element 26 is retracted within the sleeve 14 with no part of the gripping element 26 extended distally beyond the open distal end of the sleeve. In this configuration, the sleeve 14 with gripping element 26 may be advanced through the natural orifice to the tissue sought to be manipulated, such as, e.g., the appendix, gall bladder, diverticulum, etc.

Once the sleeve 14 is juxtaposed with the tissue, as shown in FIG. 4 the gripping element 26 is advanced by means of the control rod 20 out of the distal end of the sleeve 14 and into tissue 36, typically into a void that is naturally formed by the tissue. If desired, the interior of the sleeve 14 may be evacuated by appropriately operating the source 24 of pressure shown in FIG. 1 to attract the tissue 36 toward the sleeve 14, including toward the vacuum openings 16 and the open distal end of the sleeve. Evacuation also causes the tissue to collapse onto the grippers 30, with the ends of a multitude of grippers establishing anchor points to the tissue. In addition to vacuum or alternatively, the gripping element 26 may be rotated to tighten the tissue onto the grippers 30. In any case, adequate time may be allocated to permit the vacuum to collapse the tissue.

By providing multiple points of contact (e.g., multiple bristles), less damage to the tissue is effected during manipulation and furthermore, minimally traumatic disengagement of the gripping element 26 with the tissue should such become necessary is facilitated. To disengage the tissue, a positive pressure source may be actuated to pressurize the interior of the sleeve 14 and thereby urge tissue away from the sleeve and bristles.

As shown in FIG. 5 the gripping element 26 may be retracted proximally relative to the sleeve 14 to trap or wedge the tissue 36 between the grippers 30 and the interior of the sleeve 14 as shown. With the tissue thus firmly gripped, it may be manipulated as desired, e.g., the tissue may be inverted, moved, retracted, resected, etc. as appropriate for the particular procedure. Instructions may be provided on, e.g., a substrate to effect the above steps.

FIG. 6 shows that in one non-limiting implementation, a brush 40 with plural generally radially oriented and relatively rigid bristles may be engaged with a delivery tube 42 formed with plural vacuum holes 44. The diameter “D1” of the brush 40 may be 2.3 mm and the brush may be 20 mm in length. The brush 40 may slide within the tube 42 or it may be stationarily engaged with the tube 42, in which case a cover tube such as the cover tube 32 shown in FIG. 1 that can be made of, e.g., Teflon™ can be used to enclose the brush 40 during delivery to the tissue site.

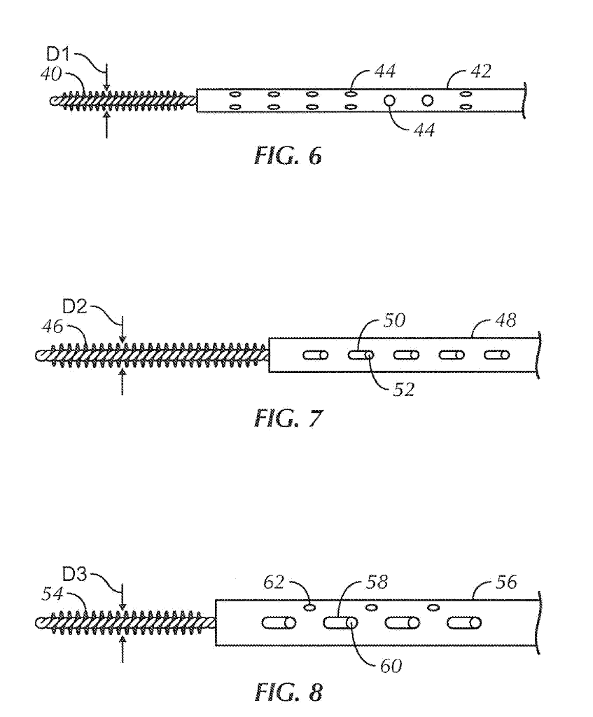

FIG. 7 shows that in another non-limiting implementation, a brush 46 with plural generally radially oriented and relatively rigid bristles may be engaged with a delivery tube 48 formed with plural elongated vacuum notches 50 that do not extend completely through the wall of the tube 48 but that terminate in respective vacuum holes 52 that do extend through the wall of the tube. The notches 50 with holes 52 may be formed in two lines on opposite sides of the tube. The diameter “D2” of the brush 46 may be 2.5 mm and the brush may be 25 mm in length. The brush 46 may slide within the tube 48 or it may be stationarily engaged with the tube 48, in which case a cover tube such as the cover tube 32 shown in FIG. 1 that can be made of, e.g., Pebax™ can be used to enclose the brush 40 during delivery to the tissue site.

FIG. 8 shows that in another non-limiting implementation, a brush 54 with plural generally radially oriented and relatively rigid bristles may be engaged with a delivery tube 56 formed with plural elongated vacuum notches 58 that do not extend completely through the wall of the tube 56 but that terminate in respective vacuum holes 60 that do extend through the wall of the tube. The notches 56 with holes 60 may be formed in two lines on opposite sides of the tube, and additional holes 62 may be formed without notches in a spiral pattern as shown. Tape may be used to cover holes that are not desired to be used to establish a vacuum. The brush can be exposed at various lengths relative to the tube. The hole pattern inhibits tissue slippage to minimize unwanted twisting of the appendix.

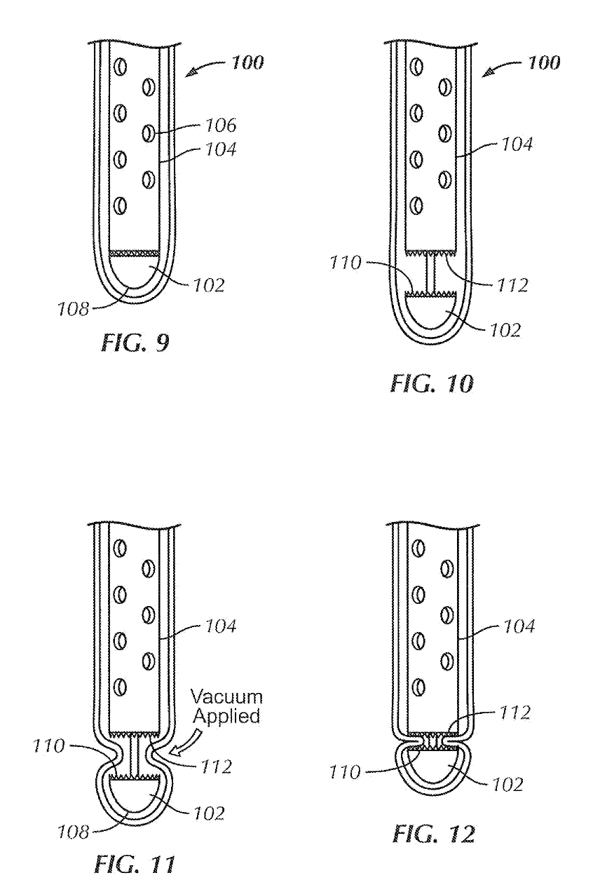

FIGS. 9-12 show an alternate system 100 in which a gripping element 102 is slidably engaged with a delivery tube 104 that may be formed with vacuum openings 106 in accordance with disclosure above. As shown, the gripping element 102 includes a rounded smooth atraumatic distal end 108 defining a flat disc-like proximal-facing surface formed with plural teeth 110. Also, the distal end of the tube 104 defines a distal-facing flat disc-like surface formed with plural tube teeth 112.

With this structure, the system 100 is advanced through a natural orifice with the gripping element 102 retracted into the tube 104 (FIG. 9) such that the teeth 110, 112 mesh, or alternatively are in substantial contact with each other. When positioned in the target tissue the gripping element 102 is advanced away from the tube 104 (FIG. 10) into the tissue and then vacuum is established in the tube 104 (FIG. 11) to attract tissue into the space between the teeth 110, 112. The gripping element 102 is then retracted proximally relative to the tube 104 (FIG. 12) to trap tissue between the teeth 110, 112. The shaft of the gripping element 102 may further include bristles in accordance with the disclosure above in addition to the structure shown in FIGS. 9-12.

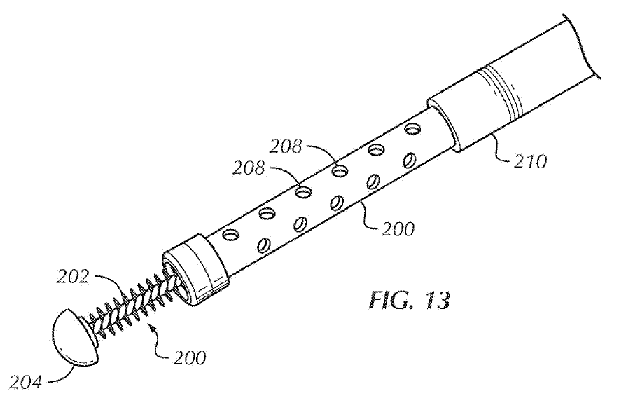

FIG. 13 illustrates a gripping element 200 with bristles 202 and atraumatic distal tip 204 that may be slidably engaged with a sleeve 206. The sleeve 206 may be formed with vacuum openings 208. A cover tube 210 such as the cover tube 32 shown in FIG. 1 can be used to enclose the bristles and/or to cover the vacuum openings 208.

While the particular BRUSH DEVICE FOR GRASPING AND MANIPULATING TISSUE are herein shown and described in detail, it is to be understood that the subject matter which is encompassed by the present invention is limited only by the claims.

What is claimed is:

1. Apparatus comprising:
a tissue gripping element housed within a tubular member for advancement through a natural orifice to tissue to be inverted pursuant to resolution of a malady associated with the tissue, the tissue gripping element being advanceable out of the tubular member into the tissue to grip the tissue, the tissue gripping element including plural discrete gripping points.

2. The apparatus of claim 1, wherein the gripping points are established by ends of respective bristles oriented generally radially relative to the tubular member.

3. The apparatus of claim 1, wherein the gripping points are established by ends of respective teeth.

4. The apparatus of claim 3, wherein the teeth are arranged on a proximal-facing transverse surface, and plural tubular member teeth are arranged on a distal-facing transverse surface of the tubular member, the tissue gripping element being distally advanceable relative to the tubular member to space the surfaces from each other and proximally retractable relative to the tubular member to trap tissue between the surfaces.

5. The apparatus of claim 1, wherein the tubular member establishes a vacuum lumen through which a vacuum can be drawn to attract tissue toward the tubular member.

6. The apparatus of claim 5, wherein tissue is attracted toward an open distal end of the tubular member when a vacuum is drawn in the tubular member.

7. The apparatus of claim 5, wherein tissue is attracted toward plural vacuum openings formed in the tubular member when a vacuum is drawn in the tubular member.

8. The apparatus of claim 7, comprising a tube advanceable over the tubular member to block at least some of the vacuum openings.

9. The apparatus of claim 1, wherein the tissue gripping element is rotatable relative to the tubular member.

10. The apparatus of claim 1, comprising a smooth rounded atraumatic surface establishing a distal end of the tissue gripping element.

11. Method comprising:
establishing a retracted configuration of a tissue manipulation device wherein a tissue gripping element is retracted entirely within a sleeve, the tissue gripping element including plural discrete gripping points;
providing instructions to advance the tissue manipulation device through a natural body orifice to tissue to be manipulated;
providing instructions to advance the tissue gripping element out of the sleeve; and
providing instructions to manipulate the tissue gripping element to grip tissue.

12. Method of claim 11, comprising providing instructions to retract the tissue gripping element toward the sleeve.

13. The method of claim 11, comprising providing instructions to establish a vacuum in the sleeve.

14. The method of claim 11, wherein the gripping points are established by ends of respective bristles oriented generally radially relative to the sleeve.

15. The method of claim 11, wherein the gripping points are established by ends of respective teeth.

16. System comprising:
a delivery tube advanceable into a natural body orifice toward tissue to be manipulated;
an elongated control rod extending from a proximal end of the tube and manipulable by a person; and
a tissue gripping element coupled to the control rod and advanceable out of a distal end of the delivery tube, the gripping element including plural individual grippers configured to engage tissue and thereby provide a means for manipulating the tissue by manipulating the control rod.

17. The system of claim 16, wherein the grippers are established by ends of respective bristles oriented generally radially relative to the delivery tube.

18. The system of claim 16, wherein the grippers are established by ends of respective teeth.

19. The system of claim 18, wherein the teeth are arranged on a proximal-facing transverse surface, and plural tube teeth are arranged on a distal-facing transverse surface of the delivery tube, the tissue gripping element being distally advanceable relative to the tube to space the surfaces from each other and proximally retractable relative to the tube to trap tissue between the surfaces.

20. The system of claim 16, wherein the tube establishes a vacuum lumen through which a vacuum can be drawn to attract tissue toward the gripping element.
